(19) United States
(12) Patent Application Publication
(10) Pub. No.: US 2017/0360443 A1
(43) Pub. Date: Dec. 21, 2017

(54) DAMAGE RESISTANT ANVIL ASSEMBLY

(71) Applicant: Covidien LP, Mansfield, MA (US)

(72) Inventor: Justin Williams, Southbury, CT (US)

(21) Appl. No.: 15/606,289

(22) Filed: May 26, 2017

Related U.S. Application Data
(60) Provisional application No. 62/352,683, filed on Jun. 21, 2016.

(51) Int. Cl. A61B 17/115; A61B 17/3205; A61B 17/00; A61B 17/068; A61B 17/072

(52) U.S. Cl. CPC A61B 17/1155; A61B 17/32053; A61B 2017/0725; A61B 2017/07214; A61B 17/068; A61B 2017/00367; A61B 17/072; A61B 2017/07257; A61B 2017/00473

(57) ABSTRACT

An anvil assembly is disclosed that includes an anvil shaft including a proximal portion and a distal portion and defining a first longitudinal bore. The proximal portion includes a plurality of flexible legs that define the first longitudinal bore dimensioned to receive a trocar of a stapling device. An anvil head assembly is secured to the distal portion of the anvil shaft and supports an annular anvil plate that a plurality of staple deforming pockets. The anvil assembly also includes a rigid collet defining a second longitudinal bore that is configured to receive the trocar of the stapling device. The rigid collet is supported within the first longitudinal bore and is positioned to prevent crushing of the plurality of flexible legs when the anvil assembly is manipulated with a grasper.

### CROSS-REFERENCE TO RELATED APPLICATIONS

This application claims the benefit of and priority to U.S. Provisional Patent Application No. 62/352,683 filed Jun. 21, 2016, the entire disclosure of which is incorporated by reference herein.

### BACKGROUND

### 1. Technical Description

The present disclosure is directed to an anvil assembly for use with a surgical stapling device. More particularly, the present disclosure is directed to an anvil assembly for a circular surgical stapling device including a stabilizing collet positioned to prevent damage to the anvil assembly.

### 2. Background of Related Art

Circular staplers are commonly used to perform a variety of surgical procedures including anastomosis procedures for joining ends of tubular tissue sections and hemorrhoidectomy procedures for treating hemorrhoids. Typically, circular staplers include a stapling device and an anvil assembly. The stapling device includes a handle assembly, a body portion extending from the handle assembly, a shell assembly including a staple cartridge, and a trocar extending from the shell assembly. The anvil assembly is releasably secured to the trocar of the stapling device and includes an anvil assembly having an anvil shaft and an anvil head assembly. The shell assembly includes a circular knife. When the circular stapler is fired, the circular knife is advanced from the shell assembly and cuts tissue as staples are ejected from the staple cartridge and formed against the anvil head assembly. In use, the stapling device and the anvil assembly are delivered to a surgical site within a patient separately and coupled to each other prior to use.

Typically, the stapling device and the anvil assembly are coupled together at the surgical site by a clinician using a grasper. More particularly, the clinician grasps the anvil shaft of the anvil assembly with the grasper and positions the anvil shaft about the trocar of the stapling device to couple the trocar to the anvil shaft. This coupling procedure takes place within a body lumen or orifice where visibility is limited.

When a clinician applies too much pressure on the anvil shaft, the anvil shaft can be damaged, e.g., crushed or deformed, such that the anvil shaft cannot be properly coupled to the stapling device. This problem is exacerbated because due to the poor visibility at the surgical site, the clinician may be unaware that the anvil shaft has been damaged and is not properly coupled to the stapling device. As such, when circular stapler is fired, the anvil assembly may become disengaged from the stapling device such that the staples are not formed in cut tissue.

Accordingly, a need exists in the surgical arts for an anvil assembly that is less susceptible to damage during attachment of the anvil assembly to the stapling device to facilitate reliable attachment of the anvil assembly to a stapling device.

### SUMMARY

In one aspect of the disclosure, an anvil assembly includes an anvil shaft defining a first longitudinal bore and an anvil head assembly. The anvil shaft has a proximal portion and a distal portion. The proximal portion includes a plurality of flexible legs that define the first longitudinal bore. The anvil head assembly is secured to the distal portion of the anvil shaft and supports an anvil plate that defines a plurality of staple deforming pockets. A stabilizing collet defines a second longitudinal bore. The collet is supported within the first longitudinal bore and is positioned to prevent damage to the plurality of flexible legs.

In another aspect of the disclosure, a surgical stapler includes a stapling device and an anvil assembly. The stapling device includes a handle assembly, a body portion that extends distally from the handle assembly, a shell assembly including a staple cartridge having a plurality of staples, and a trocar extending from the shell assembly. The anvil assembly includes an anvil shaft and an anvil head assembly. The anvil shaft has a proximal portion and a distal portion and defines a first longitudinal bore configured to receive the trocar of the stapling device. The proximal portion includes a plurality of flexible legs that defines the first longitudinal bore. The anvil head assembly is secured to the distal portion of the anvil shaft and supports an anvil plate that defines a plurality of staple deforming pockets. A stabilizing collet defines a second longitudinal bore configured to receive the trocar. The collet is supported within the first longitudinal bore and is positioned to prevent damage to the plurality of flexible legs.

In embodiments, the collet is cylindrical.

In certain embodiments, the collet is substantially rigid.

In some embodiments, the collet has a distal end including a plurality of cantilevered fingers, wherein each of the plurality of cantilevered fingers has a protrusion configured to secure the collet within the first longitudinal bore of the anvil shaft.

In certain embodiments, each of the plurality of flexible legs defines a longitudinal channel with an adjacent one of the plurality of flexible legs.

In embodiments, the anvil shaft defines a hole positioned adjacent the distal end of each of the longitudinal channels. Each of the holes is configured to receive a respective one of the protrusions.

In some embodiments, each of the holes is circular.

In certain embodiments, the anvil head assembly is pivotally secured to the anvil shaft.

In embodiments, the anvil plate is annular.

### BRIEF DESCRIPTION OF THE DRAWINGS

Various embodiments of the presently disclosed crush resistant anvil assembly are described herein below with reference to the drawings, wherein:

FIG. 1 is a side perspective view of a surgical stapler including an exemplary embodiment of the presently disclosed crush resistant anvil assembly;

FIG. 2 is an enlarged view of the indicted area of detail shown in FIG. 1;

FIG. 3 is a cross-sectional view taken along section line 3-3 of FIG. 2;

FIG. 4 is a side perspective view of the anvil assembly shown in FIG. 2;

FIG. 5 is an enlarged view of the indicated area of detail shown in FIG. 4;

FIG. 6 is a side perspective view of a collet of the anvil assembly shown in FIG. 4;

FIG. 7 is a side cross-sectional view of the collet shown in FIG. 6 and the anvil shaft of the anvil assembly shown in FIG. 4 with parts separated;

FIG. 8 is a side cross-sectional view of the collet and anvil shaft shown in FIG. 7 as the collet is slid into the anvil shaft;

FIG. 9 is a side cross-sectional view of the collet and anvil shaft shown in FIG. 8 with the collet secured within the anvil shaft; and

FIG. 10 is a side cross-sectional view of the collet and anvil shaft shown in FIG. 9 as a trocar of the stapling device is positioned within the anvil shaft.

### DETAILED DESCRIPTION OF EMBODIMENTS

Exemplary embodiments of the presently disclosed damage resistant anvil assembly will now be described in detail with reference to the drawings in which like reference numerals designate identical or corresponding elements in each of the several views. In this description, the term “proximal” is used generally to refer to that portion of the stapler that is closer to a clinician, while the term “distal” is used generally to refer to that portion of the stapler that is farther from the clinician. In addition, the term “endoscopic” is used generally to refer to procedures performed through a small incision or a cannula inserted into a patient's body including endoscopic, laparoscopic, and arthroscopic procedures. Finally, the term clinician is used generally to refer to medical personnel including doctors, nurses, and support personnel.

The presently disclosed anvil assembly includes an anvil head assembly, an anvil shaft, and a stabilizing collet. In embodiments, the stabilizing collet may be formed of a substantially rigid material. Alternately, other materials of construction that provide support to the anvil shaft are envisioned. The anvil shaft includes a plurality of flexible legs that flex outwardly in response to insertion of a trocar of a surgical stapling device into the anvil shaft to releasably couple the anvil shaft to the trocar. The collet is received within a longitudinal bore defined by the flexible legs of the anvil shaft at a location to support the flexible legs and minimize the likelihood of damage to the anvil shaft caused by engagement of the anvil shaft with a grasper. The collet is also positioned in a location not to interfere with flexing of the legs during coupling of the anvil shaft to the trocar of the stapling device.

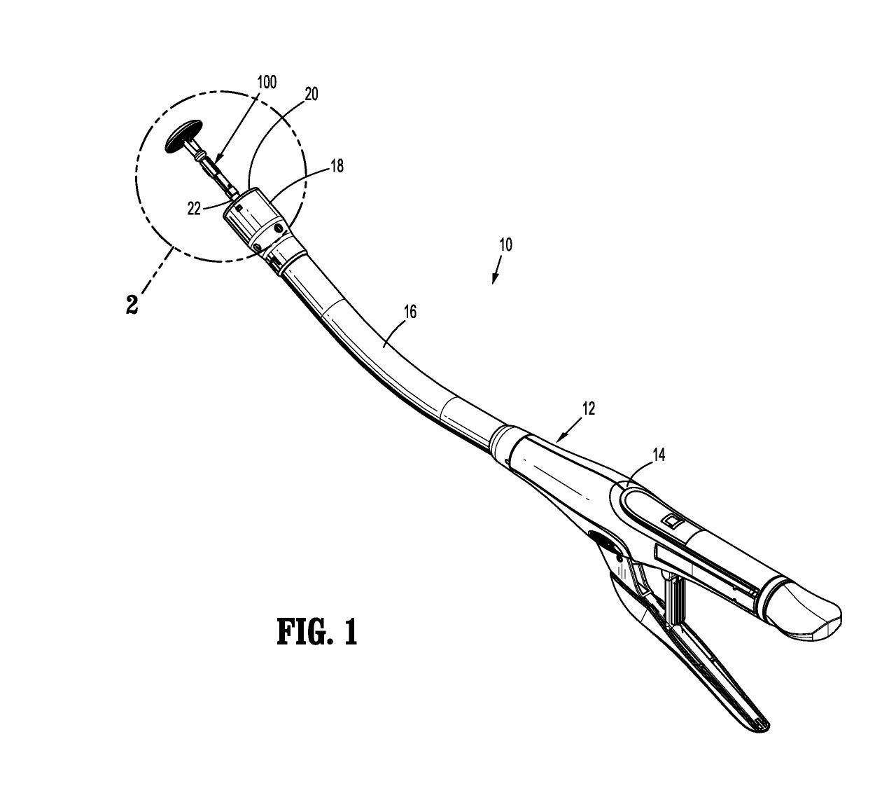

FIG. 1 illustrates a manually powered surgical stapler 10 including a stapling device 12 supporting an exemplary embodiment of anvil assembly 100. The stapling device 12 includes a handle assembly 14, a body portion 16 that extends distally from the handle portion 14, and a shell assembly 18 that supports a staple cartridge 20. The staple cartridge 20 supports a plurality of staples (not shown) that are arranged in an annular configuration within the shell assembly 18. The stapling device 12 also includes a trocar 22 that extends from the distal end of the body portion 16 through the shell assembly 18. The trocar 22 is configured to releasably engage the anvil assembly 100 as described in further detail below. For a more detailed description of a suitable stapling device, see U.S. Pat. Nos. 7,234,624, 7,364,060 and 7,857,187 (“the incorporated patents”) which are incorporated herein by reference in their entirety.

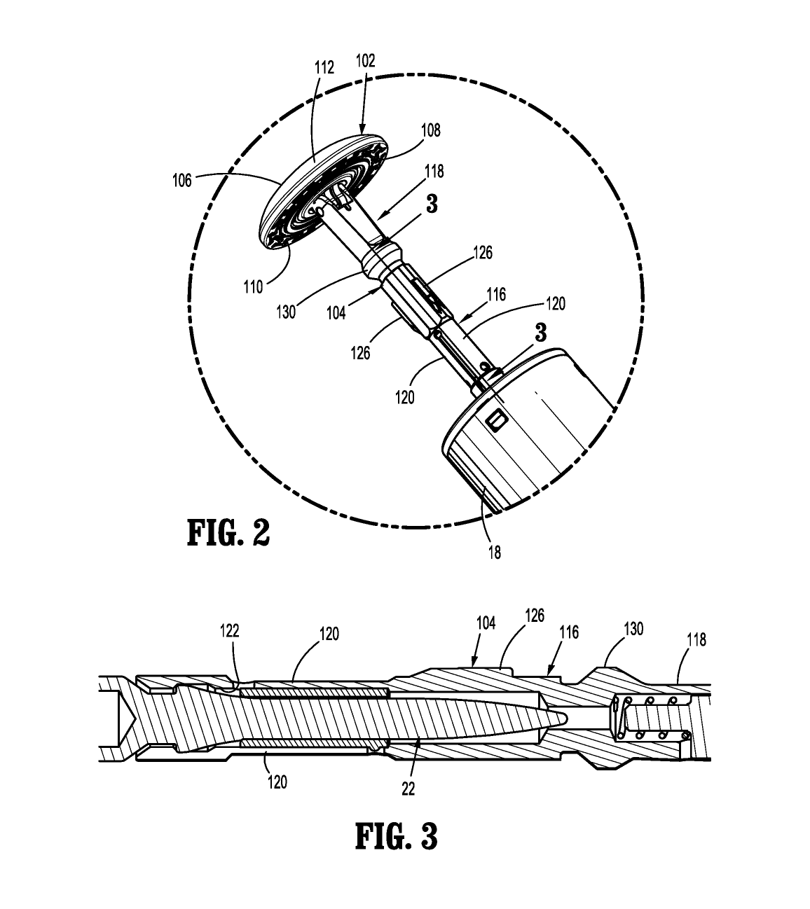

Referring also to FIGS. 2-4, the anvil assembly 100 includes an anvil head assembly 102 and an anvil shaft 104. Although not specifically described in this application, the anvil head assembly 102 can be pivotally or fixedly attached to the anvil shaft 104. Examples of pivotally attached anvil head assemblies are described in the incorporated patents.

The anvil head assembly 102 includes a housing 106 that supports an anvil plate 108 (FIG. 2) and a cut ring assembly 110. The housing 106 has a smoothly curved distal surface 112 that facilitates atraumatic entry of the anvil assembly 100 into and through a body orifice or lumen. A proximal side of the housing 106 defines a cavity (not shown) that is configured to receive the anvil plate 108 and the cut ring assembly 110. For a more detailed description of the components of the anvil head assembly 102, see the incorporated patents.

The anvil shaft 104 includes a longitudinal body portion 116 that includes a tubular portion 118 and a plurality of flexible legs 120 that extend proximally from the tubular portion 118. Each of the flexible legs 120 has a semi-cylindrical configuration such that the legs 120 cooperate to define a longitudinal bore 122 (FIG. 3) that is dimensioned to receive the trocar 22 of the stapling device 12 (FIG. 1) when the anvil assembly 100 is secured to the stapling device 12. The bore 122 extends from the proximal end of the flexible legs 120 at least partially into the tubular portion 118 of the anvil shaft 104.

In embodiments, the anvil shaft 104 may include a plurality of splines 126 positioned about the anvil shaft 104. As is known in the art, the splines 126 mate with recesses (not shown) defined within the shell assembly 16 FIG. 2) of the surgical stapling device 12 to properly orient the staple cartridge 20 in relation to the anvil plate 108 of the anvil assembly 100 when the anvil assembly 100 and the shell assembly 18 are approximated. The anvil shaft 104 may also include one or more stabilization rings 130 (only one is shown) positioned about the anvil shaft 104 at a position to engage the shell assembly 16 when the anvil assembly 100 and the shell assembly 18 are approximated to provide added stability to the anvil assembly 100. For a more detailed description of an anvil assembly including a stabilization ring, see U.S. Pat. No. 8,424,535 which is incorporated herein by reference in its entirety. Although the splines 126 and the stabilization ring 130 are shown to be formed integrally with the anvil shaft 104, it is contemplated the either or both could be formed separately from the anvil shaft 104 and secured to the anvil shaft 104 using any known fastening technique including welding, crimping gluing or the like.

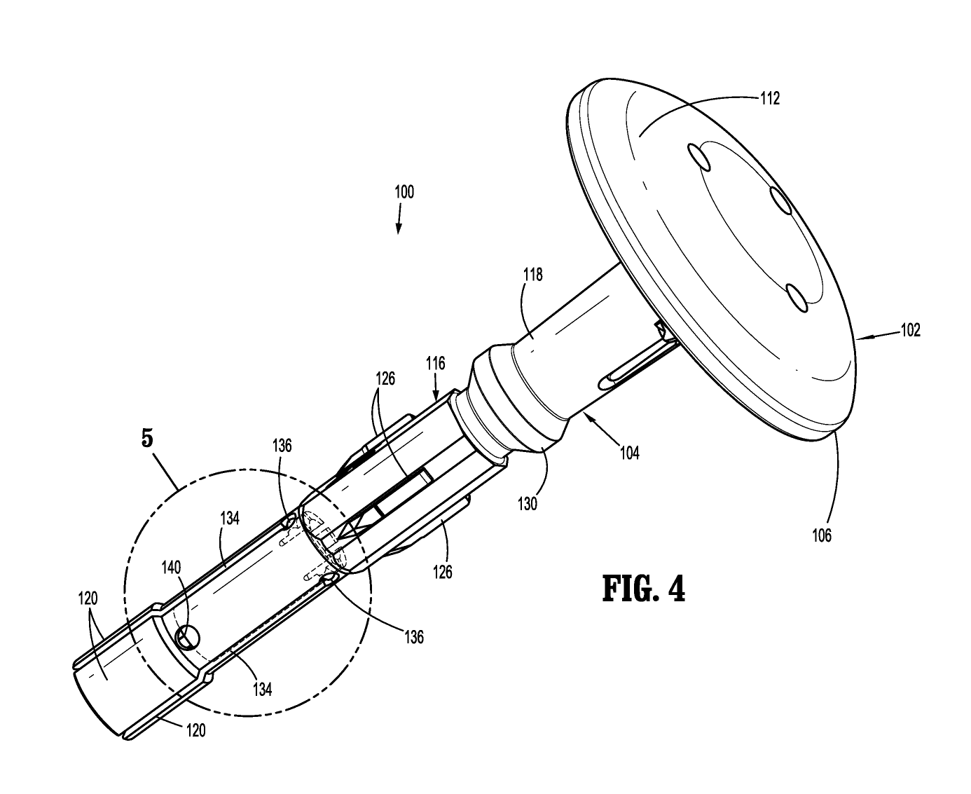

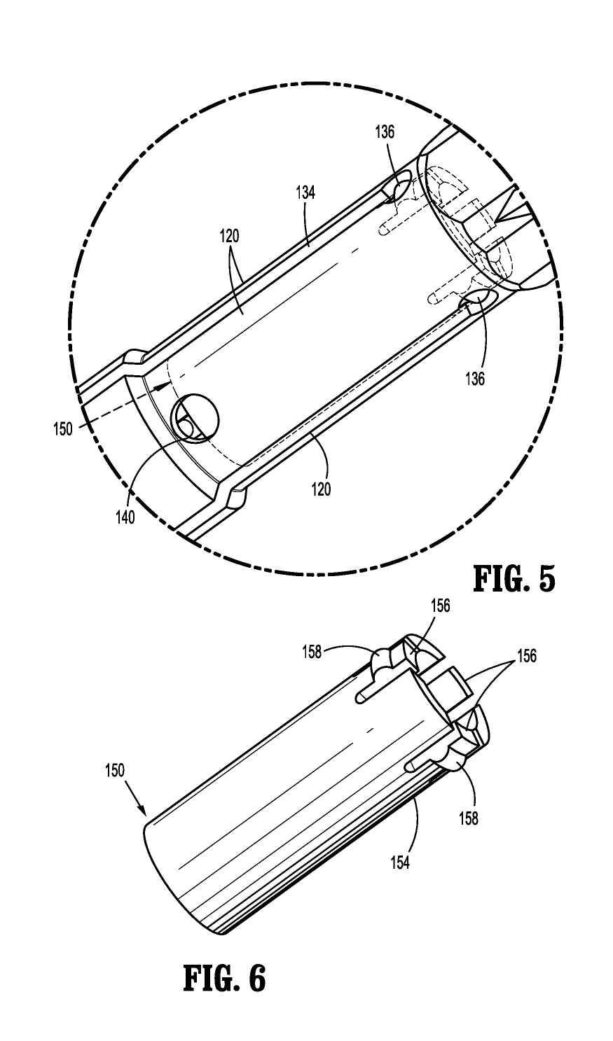

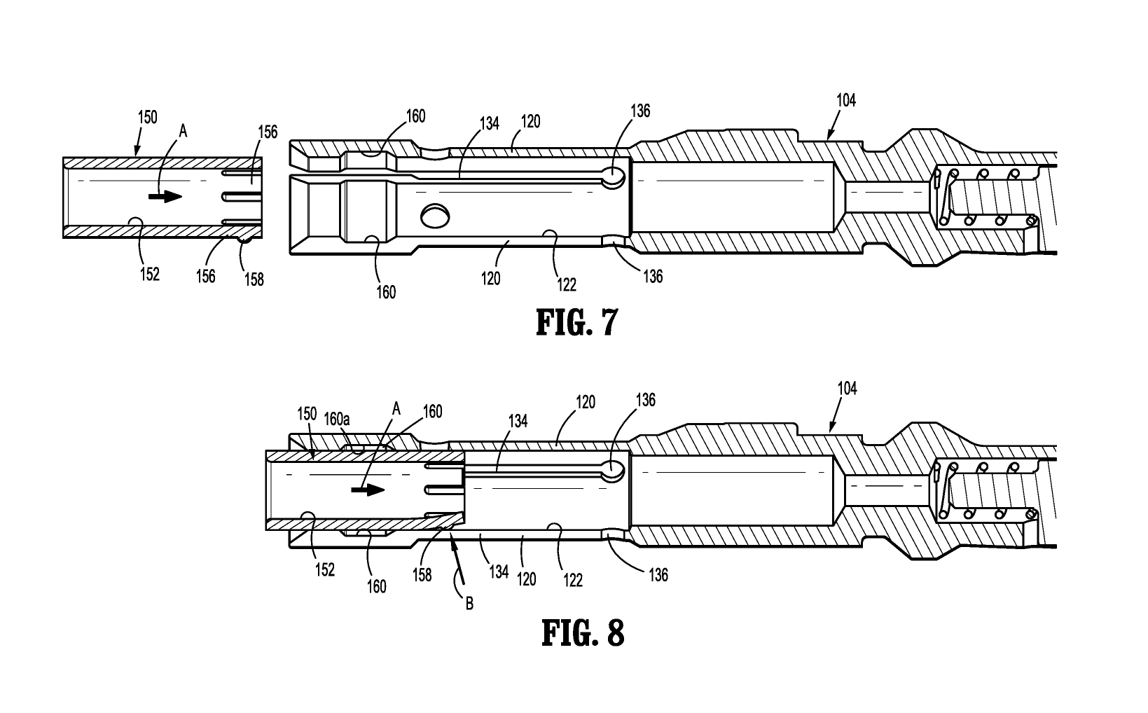

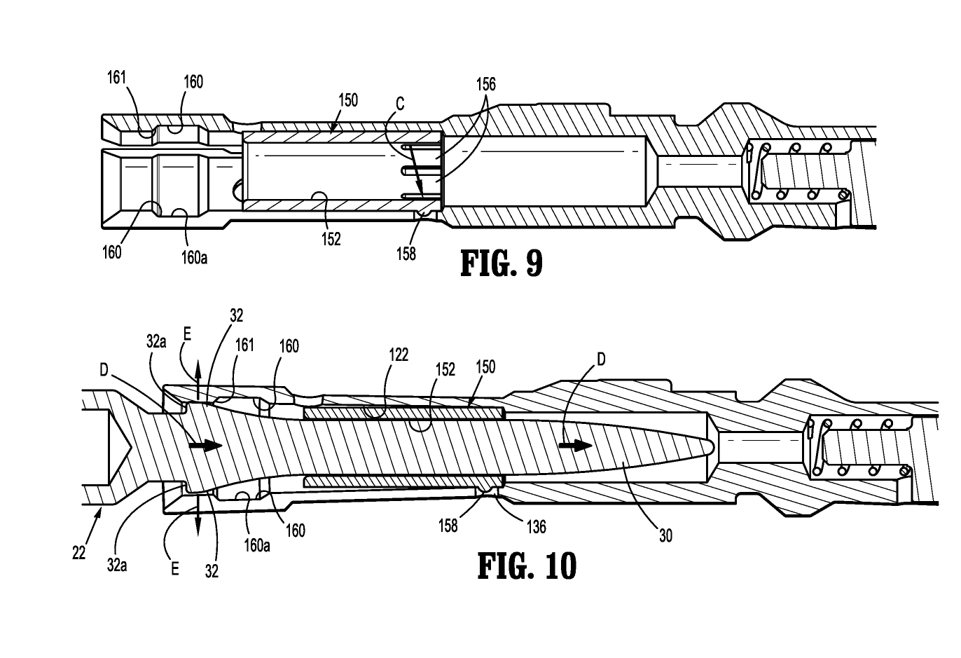

Referring to FIGS. 4 and 5, each of the flexible legs 120 of the anvil shaft 104 defines a longitudinal channel 134 with an adjacent leg 120. Each longitudinal channel 134 includes an enlarged cutout or hole 136 formed at the distal end of the longitudinal channel 134. The holes 136 are configured to secure a collet 150 within the longitudinal bore 122 of the anvil shaft 104. In embodiments, the hole 136 is substantially circular although other configurations are envisioned. One or more of the flexible legs 120 may also include a bore 140 which is configured to receive a suture or the like (not shown). The suture can be used to allow a clinician to retrieve or position the anvil assembly 100 from or within a surgical site. The proximal end of each of the flexible legs 120 has an inner surface that defines a recess 160 (FIG. 7) such that the recesses 160 collectively define an annular recess 160a (FIG. 9). The annular recess 160a facilitates releasable engagement of the anvil assembly 100 to the stapling device 12.

Referring also to FIG. 6, the collet 150 may be substantially rigid and is positioned within the longitudinal bore 122 defined by the anvil shaft 104. The collet 150 is substantially cylindrical and defines a longitudinal bore 152 (FIG. 7) that is dimensioned to receive the trocar 22 (FIG. 10). A distal portion 154 of the collet 150 includes a plurality of cantilevered fingers 156. Each of the fingers 156 includes a protrusion 158 that is dimensioned and configured to be received in a respective one of the holes 136 (FIG. 5) formed in the anvil shaft 104 as described in further detail below.

Referring to FIGS. 7-9, in order to assemble the collet 150 within the anvil shaft 104, the distal end of the collet 150 is inserted into the proximal end of the longitudinal bore 122 of the anvil shaft 104 and slid distally in the direction indicated by arrow “A” in FIGS. 7 and 8. The collet 150 is positioned to align the protrusions 158 with the longitudinal channels 134 positioned between the flexible legs 120. When the protrusions 158 engage an inner wall of the flexible legs 120, the fingers 156 are deflected inwardly in the direction indicated by arrow “B” in FIG. 8 to facilitate passage of the collet 150 through the longitudinal bore 122. When the protrusions 158 are moved into alignment with the holes 136, the fingers 156 spring outwardly in the direction indicated by arrow “C” in FIG. 9 to move the protrusions 158 into the holes 136 to secure the collet 150 within the longitudinal bore 122.

Referring to FIG. 10, the trocar 22 includes a pointed distal end 30 and an enlarged proximal portion 32 that defines a shoulder 32a. As known in the art, the proximal end of the trocar 22 is secured to an approximation mechanism (not shown) of the stapling device 12 (FIG. 1) to facilitate movement of the trocar 22 between retracted and advanced positions. When the trocar 22 is inserted into the longitudinal bore 122 of the anvil shaft 104 and the longitudinal bore 152 of the collet 150 in the direction indicated by arrow “D” in FIG. 10, the enlarged proximal portion 32 of the trocar 22 engages a proximal end of the flexible legs 120 of the anvil shaft 104 to urge the flexible legs 120 outwardly in the direction indicated by arrows “E”. When the enlarged proximal portion 32 of the trocar 22 is moved distally in the direction indicated by arrow “D” into alignment with the recess 160 defined at the proximal end of the flexible legs 120, the flexible legs 120 return to their undeformed configuration to receive the enlarged proximal portion 32 of the trocar 22. When the enlarged proximal portion 32 is received within the recess 160, the shoulder 32a on the enlarged proximal portion 32 of the trocar 32 engages a proximal wall 161 defining the recess 160 to secure the anvil shaft 104 to the trocar 22.

During an endoscopic surgical procedure, the anvil assembly 100 is grasped with a grasper (not shown) that is inserted through a small incision in the skin to position the trocar 22 within the longitudinal bore 122 of the anvil shaft 104 and secure the anvil assembly 100 to the trocar 22 of the surgical stapling device 12. The collet 150 is positioned within the longitudinal bore 122 of the anvil shaft 104 and extends from a distal end of the flexible legs 120 towards the proximal end of the flexible legs 120 to support the flexible legs 120 and inhibit radial compression or other deformation of the flexible legs 120 that may result from pressure applied to the flexible legs 120 by a manipulating instrument (not shown). Collet 150 may be formed from any suitable, medical grade material having a stiffness to perform the functions described herein. Suitable materials include, for example, stainless steel or nylon. The collet 150 is secured within the longitudinal bore 122 of the anvil shaft 104 in a position that does not interfere with outward flexing of the flexible legs 120 and, thus, allows the anvil assembly 100 to be readily connected to the trocar 22.

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described embodiments. Accordingly, the disclosure is not to be limited by what has been particularly shown and described, except as indicated by the appended claims.

What is claimed is:

1. An anvil assembly comprising:
an anvil shaft defining a first longitudinal bore, the anvil shaft having a proximal portion and a distal portion, the proximal portion including a plurality of flexible legs that define the first longitudinal bore;
an anvil head assembly secured to the distal portion of the anvil shaft, the anvil head assembly supporting an anvil plate defining a plurality of staple deforming pockets; and
a stabilizing collet defining a second longitudinal bore, the stabilizing collet supported within the first longitudinal bore and being positioned to prevent damage to the plurality of flexible legs.

2. The anvil assembly of claim 1, wherein the collet is cylindrical.

3. The anvil assembly of claim 2, wherein the collet has a distal end including a plurality of cantilevered fingers, each of the plurality of cantilevered fingers having a protrusion configured to secure the collet within the first longitudinal bore of the anvil shaft.

4. The anvil assembly of claim 3, wherein each of the plurality of flexible legs defines a longitudinal channel with an adjacent one of the plurality of flexible legs.

5. The anvil assembly of claim 4, further including a hole formed in the anvil shaft adjacent the distal end of each of the longitudinal channels, each of the holes being configured to receive a respective one of the protrusions.

6. The anvil assembly of claim 5, wherein each of the holes is circular.

7. The anvil assembly of claim 1, wherein the anvil head assembly is pivotally secured to the anvil shaft.

8. The anvil assembly of claim 1, wherein the anvil plate is annular.

9. The anvil assembly of claim 1, wherein the collet is rigid.

10. A surgical stapler comprising:
a stapling device including a handle assembly, a body portion that extends distally from the handle assembly, a shell assembly including a staple cartridge having a plurality of staples, and a trocar extending from the shell assembly; and
an anvil assembly including an anvil shaft and an anvil head assembly, the anvil shaft defining a first longitudinal bore configured to receive the trocar, the anvil shaft having a proximal portion and a distal portion, the proximal portion including a plurality of flexible legs that define the first longitudinal bore, the anvil head assembly secured to the distal portion of the anvil shaft and supporting an anvil plate defining a plurality of staple deforming pockets, and a stabilizing collet defining a second longitudinal bore configured to receive the trocar, the collet supported within the first longitudinal bore and being positioned to prevent damage to the plurality of flexible legs.

11. The surgical stapler of claim 10, wherein the collet is cylindrical.

12. The surgical stapler of claim 11, wherein the collet has a distal end including a plurality of cantilevered fingers, each of the plurality of cantilevered fingers having a protrusion configured to secure the collet within the first longitudinal bore of the anvil shaft.

13. The surgical stapler of claim 12, wherein the collet has a distal end including a plurality of cantilevered fingers, each of the plurality of cantilevered fingers having a protrusion configured to secure the collet within the first longitudinal bore of the anvil shaft.

14. The surgical stapler of claim 13, wherein each of the plurality of flexible legs defines a longitudinal channel with an adjacent one of the plurality of flexible legs.

15. The surgical stapler of claim 14, further including a hole formed in the anvil shaft adjacent a distal end of each of the longitudinal channels, each of the holes being configured to receive a respective one of the protrusions.

16. The surgical stapler of claim 15, wherein each of the holes is circular.

17. The surgical stapler of claim 16, wherein the anvil head assembly is pivotally secured to the anvil shaft.

18. The surgical stapler of claim 11, wherein the anvil plate is annular.

19. The anvil assembly of claim 10, wherein the collet is rigid.
